(19) United States
(12) Patent Application Publication
(10) Pub. No.: US 2010/0016722 A1
(43) Pub. Date: Jan. 21, 2010

(54) FORMATION OF AN ELASTIC IMAGE IN AN ULTRASOUND SYSTEM

(75) Inventors: Dong Kuk SHIN, Seoul (KR); Jong Sik Kim, Seoul (KR)

Correspondence Address: JONES DAY, 222 EAST 41ST ST, NEW YORK, NY, 10017, US

(73) Assignee: Medison Co., Ltd.

(21) Appl. No.: 12/501,357

(22) Filed: Jul. 10, 2009

(30) Foreign Application Priority Data
Jul. 16, 2008 (KR) 10-2008-0068909

(51) Int. Cl. A61B 8/14

(52) U.S. Cl. 600/443

(57) ABSTRACT

Embodiments for forming an elastic image in an ultrasound system are disclosed. In one embodiment, an ultrasound data acquisition unit is configured to transmit/receive ultrasound signals to/from a target object to thereby output first ultrasound data. The ultrasound data acquisition unit is configured to transmit/receive ultrasound signals to/from the target object while applying pressure to the target object to thereby output second ultrasound data. A frame image data forming unit is configured to form a first frame image data based on the first ultrasound data. The frame image data forming unit is configured to form a second frame image data based on the second ultrasound data. A processing unit is configured to calculate strain data based on the first and second frame image data. The processing unit is configured to set a reference value based on the strain data. The processing unit is configured to form an elastic image based on the reference value. A control unit is coupled to the ultrasound data acquisition unit, the frame image data forming unit and the processing unit.

The present application claims priority from Korean Patent Application No. 10-2008-0068909 filed on Jul. 16, 2008, the entire subject matter of which is incorporated herein by reference.

### TECHNICAL FIELD

The present disclosure generally relates to ultrasound systems, and more particularly to the formation of an elastic image in an ultrasound system.

### BACKGROUND

Recently, an ultrasound system has been extensively used in the medical field due to its non-invasive and non-destructive nature. Modern high-performance ultrasound imaging systems and techniques are commonly used to produce two dimensional ultrasound images and three-dimensional ultrasound images of internal features of patients.

Generally, the ultrasound image is displayed in a Brightness mode (B-mode) by using reflectivity caused by an acoustic impedance difference between the tissues of a target object. However, if the reflectivity of the target object is hardly different from those of the neighboring tissues such as tumor, cancer or the like, then it is not easy to recognize the target object in the B-mode image. Further, an ultrasound elastic imaging technology has been developed to display an image of the target object by using mechanical characteristics of the target object. Such technology is very helpful for diagnosing lesions such as cancers. The tumor or cancer is relatively stiffer than the neighboring tissues. Thus, when pressure is uniformly applied, a variation of the tumor or cancer is typically smaller than those of the neighboring tissues.

The elasticity of a tissue is measured by using ultrasound data obtained before and after application of the pressure to the tissue. A compression plate mounted on an ultrasound probe may be used to apply the pressure to the tissue. A user may press the compression plate on the target object, thereby applying the pressure to the tissues of the target object. In such a case, strain data in the tissues may be varied according to the pressure applied by the user. Thus, the video quality of an elastic image may be changed according to the pressure applied to the tissue.

### SUMMARY

Embodiments for forming an elastic image in an ultrasound system are disclosed herein. In one embodiment, by way of non-limiting example, an ultrasound system comprises: an ultrasound data acquisition unit configured to transmit/receive ultrasound signals to/from a target object to thereby output first ultrasound data and to transmit/receive ultrasound signals to/from the target object while applying pressure to the target object to thereby output second ultrasound data; a frame image data forming unit configured to form a first frame image data based on the first ultrasound data, the frame image data forming unit being further configured to form a second frame image data based on the second ultrasound data; a processing unit configured to calculate strain data based on the first and second frame image data and to set a reference value based on the strain data, the processing unit being further configured to form an elastic image based on the reference value; and a control unit coupled to the ultrasound data acquisition unit, the frame image data forming unit and the processing unit, the control unit being configured to control transmission/reception of the ultrasound signals and formation of the first and second frame image data.

In another embodiment, there is provided a method of forming an elastic image in an ultrasound system, comprising: a) transmitting/receiving by using an ultrasound data acquisition unit within the ultrasound system ultrasound signals to/from a target object to thereby output first ultrasound data; b) forming by using a processing unit within the ultrasound system a first frame image data based on the first ultrasound data; c) transmitting/receiving by using the ultrasound data acquisition unit within the ultrasound system ultrasound signals to/from the target object while applying pressure to the target object to thereby output second ultrasound data; d) forming by using the processing unit within the ultrasound system a second frame image data based on the second ultrasound data; and e) calculating by using the processing unit within the ultrasound system strain data based on the first and second frame data, setting a reference value based on the strain data, and forming an elastic image based on the reference value.

In yet another embodiment, there is provided a computer readable medium comprising computer executable instructions configured to perform the following acts: a) transmitting/receiving ultrasound signals to/from a target object to thereby output first ultrasound data; b) forming a first frame image data based on the first ultrasound data; c) transmitting/receiving ultrasound signals to/from the target object while applying pressure to the target object to thereby output second ultrasound data; d) forming a second frame image data based on the second ultrasound data; and e) calculating strain data based on the first and second frame data, setting a reference value based on the strain data, and forming an elastic image based on the reference value.

The Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a block diagram showing an illustrative embodiment of an ultrasound data acquisition unit.

FIG. 3 is a block diagram showing an illustrative embodiment of a processing unit.

FIG. 4 is a schematic diagram showing an example of a histogram.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

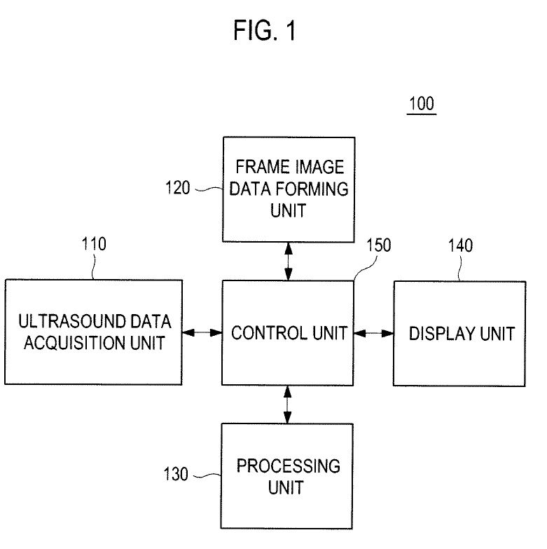

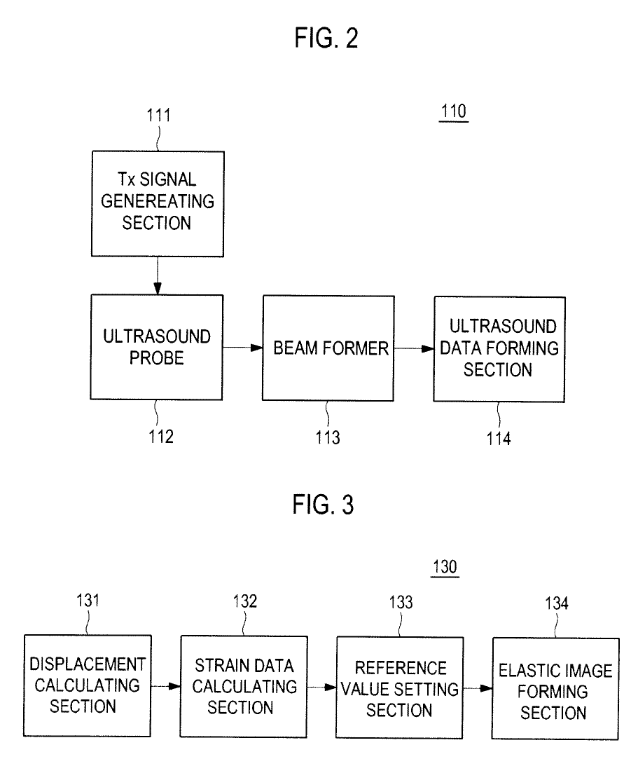

Referring to FIG. 1, an ultrasound system 100 in accordance with an illustrative embodiment is shown. As depicted therein, the ultrasound system 100 may include an ultrasound data acquisition unit 110. The ultrasound data acquisition unit 110 may be operable to transmit/receive ultrasound signals to/from a target object to thereby output ultrasound data. The ultrasound data acquisition unit 110 may include a transmit (Tx) signal generating section 111, as shown in FIG. 2.

Referring to FIG. 2, the Tx signal generating section 111 may be operable to generate first Tx signals before applying pressure to the target object. The Tx signal generating section 111 may be further operable to generate second Tx signals while applying the pressure to the target object.

The ultrasound data acquisition unit 110 may further include an ultrasound probe 112 containing a plurality of elements for reciprocally converting between ultrasound signals and electrical signals. The ultrasound probe 112 may be operable to transmit ultrasound signals into the target object in response to the first Tx signals. The ultrasound probe 112 may be further operable to receive echo signals reflected from the target object to thereby output first received signals. The received signals may be analog signals. The ultrasound probe 112 may be further operable to transmit ultrasound signals into the target object in response to the second Tx signals. The ultrasound probe 112 may be further operable to receive echo signals reflected from the target object to thereby output second received signals.

The ultrasound data acquisition unit 110 may further include a beam former 113. The beam former 113 may be operable to convert the first received signals into first digital signals. The beam former 113 may be further operable to apply delays to the first digital signals in consideration of distance between the elements and focal points to thereby output first digital receive-focused signals. The beam former 113 may be further operable to convert the second received signals into second digital signals. The beam former 113 may be further operable to apply delays to the second digital signals in consideration of distance between the elements and focal points to thereby output second digital receive-focused signals.

The ultrasound data acquisition unit 110 may further include an ultrasound data forming section 114. The ultrasound data forming section 114 may be operable to form first ultrasound data based on the first digital receive-focused signals. The ultrasound data forming section 114 may be further operable to form second ultrasound data based on the second digital receive-focused signals.

Referring back to FIG. 1, the ultrasound system 100 may further include a frame image data forming unit 120 that may be operable to form frame image data. The frame image may include a brightness mode (B-mode) image represented with gray scales based on reflectivity of the ultrasound signals in the target object. The frame image data forming unit 120 may be operable to form first frame image data of a first frame image based on the first ultrasound data. The frame image data forming unit 120 may be further operable to form second frame image data of a second frame image based on the second ultrasound data.

The ultrasound system 100 may further include a processing unit 130 that may be operable to form an elastic image. The processing unit 130 may include a displacement calculating section 131, as shown in FIG. 3.

Referring to FIG. 3, the displacement calculating section 131 may be operable to calculate displacements between the first and second frame image data. The displacement may be calculated on pixel-by-pixel or block-by-block basis between the first and second frame images by using the first and second frame image data. Also, the displacement may be calculated by using a cross-correlation or an auto-correlation.

The processing unit 130 may further include a strain data calculating section 132. The strain data calculating section 132 may be operable to calculate strain data based on the displacements.

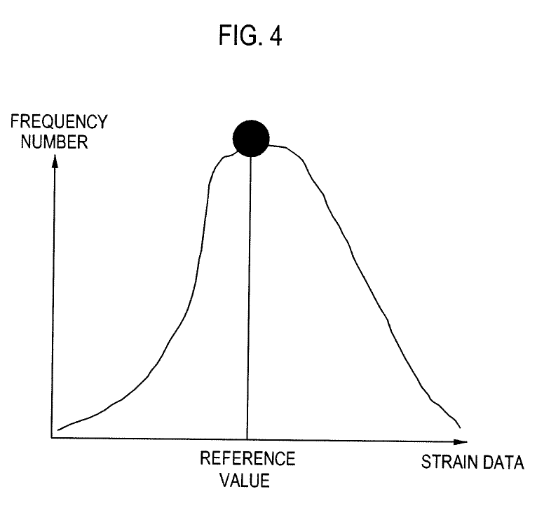

The processing unit 130 may further include a reference value setting section 133 that may be operable to set a reference value. The reference value may include one of a maximum value of frequency numbers of numerical values of the strain data and a mean value of the numerical values of the strain data. In one embodiment, the reference value setting section 133 may be operable to normalize the strain data to thereby form a histogram by using the strain data, as shown in FIG. 4. The reference value setting section 133 may be further operable to analyze the histogram to thereby detect a maximum value of frequency numbers of the numerical values of the strain data. The reference value setting section 133 may be operable to set the detected maximum value as the reference value. In one embodiment, the reference value setting section 133 may be operable to calculate a mean value of the numerical values of the strain data. The reference value setting section 133 may be further operable to set the mean value as the reference value.

The processing unit 130 may further include an elastic image forming section 134. The elastic image forming section 134 may be operable to form an elastic image based on the reference value. In one embodiment, the elastic image forming section 134 may be operable to detect strain data that are less than the reference value. The elastic image forming section 134 may be further operable to form the elastic image based on the detected strain data. The elastic image may be an elastic image of a stiff tissue (e.g., lesion). In one embodiment, the elastic image forming section 134 may be operable to detect strain data that are greater than the reference value. The elastic image forming section 134 may be further operable to form the elastic image based on the detected strain data. The elastic image may be an elastic image of a soft tissue. In one embodiment, the elastic image forming section 134 may be operable to detect strain data that are less and greater than the reference value. The elastic image forming section 134 may be further operable to form the elastic image based on the detected strain data.

Referring back to FIG. 1, the ultrasound system 100 may further include a display unit 140. The display unit 140 may be operable to display the elastic image formed in the processing unit 130.

The ultrasound system 100 may further include a control unit 150. The control unit 150 may be operable to control the transmission/reception of the ultrasound signals and formation of the ultrasound data in the ultrasound data acquisition unit 110. The control unit 150 may be further operable to control the formation of first and second frame image data in the frame image data forming unit 120. The control unit 150 may be further operable to control the formation of the elastic image in the processing unit 130. The control unit 150 may be further operable to control the display of the elastic image.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
an ultrasound data acquisition unit configured to transmit/receive ultrasound signals to/from a target object to thereby output first ultrasound data and to transmit/receive ultrasound signals to/from the target object while applying a pressure to the target object to thereby output second ultrasound data;
a frame image data forming unit configured to form a first frame image data based on the first ultrasound data and to form a second frame image data based on the second ultrasound data;
a processing unit configured to calculate strain data based on the first and second frame image data and to set a reference value based on the strain data, the processing unit being further configured to form an elastic image based on the reference value; and
a control unit coupled to the ultrasound data acquisition unit, the frame image data forming unit and the processing unit, the control unit being configured to control transmission/reception of the ultrasound signals and formation of the first and second frame image data.

2. The ultrasound system of claim 1, wherein the reference value includes one of a maximum value of frequency numbers of numerical values of the strain data and a mean value of the numerical values of the strain data.

3. The ultrasound system of claim 2, wherein the processing unit comprises:
a displacement calculating section configured to calculate displacements between the first and second frame image data;
a strain data calculating section configured to calculate the strain data based on the displacements;
a reference value setting section configured to set the reference value based on the strain data; and
an elastic image forming section configured to form the elastic image based on the reference value.

4. The ultrasound system of claim 3, wherein the reference value setting section is configured to detect the maximum value of frequency numbers of the numerical values of the strain data and to set the detected maximum value as the reference value.

5. The ultrasound system of claim 3, wherein the reference value setting section is configured to calculate the mean value of the numerical values of the strain data and to set the mean value as the reference value.

6. The ultrasound system of claim 3, wherein the elastic image forming section is configured to detect strain data that are less than the reference value and to form the elastic image based on the detected strain data.

7. The ultrasound system of claim 3, wherein the elastic image forming section is configured to detect strain data than are greater than the reference value and to form the elastic image based on the detected strain data.

8. The ultrasound system of claim 3, wherein the elastic image forming section is configured to detect strain data that are less and greater than the reference value and to form the elastic image based on the detected strain data.

9. A method of forming an elastic image in an ultrasound system, comprising:
a) transmitting/receiving by using an ultrasound data acquisition unit within the ultrasound system ultrasound signals to/from a target object to thereby output first ultrasound data;
b) forming by using a processing unit within the ultrasound system a first frame image data based on the first ultrasound data;
c) transmitting/receiving by using the ultrasound data acquisition unit within the ultrasound system ultrasound signals to/from the target object while applying pressure to the target object to thereby output second ultrasound data;
d) forming by using the processing unit within the ultrasound system a second frame image data based on the second ultrasound data; and
e) calculating by using the processing unit within the ultrasound system strain data based on the first and second frame data, setting a reference value based on the strain data and forming an elastic image based on the reference value.

10. The method of claim 9, the reference value includes one of a maximum value of frequency numbers of numerical values of the strain data and a mean value of the numerical values of the strain data.

11. The method of claim 10, wherein the step e) comprises:
e1) calculating displacements between the first and second frame image data;
e2) calculating the strain data based on the displacements;
e3) setting the reference value based on the strain data; and
e4) forming the elastic image based on the reference value.

12. The method of claim 11, the step e3) comprises:
detecting the maximum value of frequency numbers of the numerical values of the strain data; and
setting the detected maximum value as the reference value.

13. The method of claim 11, wherein the step e3) comprises:
calculating the mean value of the numerical values of the strain data; and
setting the mean value as the reference value.

14. The method of claim 11, wherein the step e4) comprises:
detecting strain data that are less than the reference value; and
forming the elastic image based on the detected strain data.

15. The method of claim 11, wherein the step e4) comprises:
detecting strain data that are greater than the reference value; and
forming the elastic image based on the detected strain data.

16. The method of claim 11, wherein the step e4) comprises:
detecting strain data that are less and greater than the reference value; and
forming the elastic image based on the detected strain data.

17. A computer readable medium comprising computer executable instructions configured to perform following acts:
a) transmitting/receiving ultrasound signals to/from a target object to thereby output first ultrasound data;
b) forming a first frame image data based on the first ultrasound data;
c) transmitting/receiving ultrasound signals to/from the target object while applying pressure to the target object to thereby output second ultrasound data;
d) forming a second frame image data based on the second ultrasound data; and
e) calculating strain data based on the first and second frame data, setting a reference value based on the strain data and forming an elastic image based on the reference value.

18. The computer readable medium of claim 17, wherein the reference value includes one of a maximum value of frequency numbers of numerical values of the strain data and a mean value of the numerical values of the strain data.

19. The computer readable medium of claim 18, wherein the act e) comprises:
e1) calculating displacements between the first and second frame image data;
e2) calculating the strain data based on the displacements;
e3) setting the reference value based on the strain data; and
e4) forming the elastic image based on the reference value.

20. The computer readable medium of claim 19, wherein the act e3) comprises:
detecting the maximum value of frequency numbers of the numerical values of the strain data; and
setting the detected maximum value as the reference value

21. The computer readable medium of claim 19, wherein the act e3) comprises:
calculating the mean value of the numerical values of the strain data; and
setting the mean value as the reference value.

22. The computer readable medium of claim 19, wherein the act e4) comprises:
detecting strain data that are less than the reference value; and
forming the elastic image based on the detected strain data.

23. The computer readable medium of claim 19, wherein the act e4) comprises:
detecting strain data that are greater than the reference value; and
forming the elastic image based on the detected strain data.

24. The computer readable medium of claim 19, wherein the act e4) comprises:
detecting strain data that are less and greater than the reference value; and
forming the elastic image based on the detected strain data.
